SEQUENCE LISTING (28 sequences)

SEQ ID NO: 1 - RNA, 22 nt, synthetic construct
  source 1..22: mol_type = other RNA; note = bta-miR-2285bn
aaaacctgaatgaactttttgt

SEQ ID NO: 2 - RNA, 23 nt, synthetic construct
  source 1..23: mol_type = other RNA; note = bta-miR-2285ce
gaaaacctgaatgaactttttga

SEQ ID NO: 3 - RNA, 21 nt, synthetic construct
  source 1..21: mol_type = other RNA; note = bta-miR-10225b
tcgagcctgacagatcacaca

SEQ ID NO: 4 - RNA, 20 nt, synthetic construct
  source 1..20: mol_type = other RNA; note = bta-miR-369-5p
atcgaccgtgttatattcgc

SEQ ID NO: 5 - RNA, 21 nt, synthetic construct
  source 1..21: mol_type = other RNA; note = bta-miR-219-3p
agaattgtggctggacatctg

SEQ ID NO: 6 - RNA, 22 nt, synthetic construct
  source 1..22: mol_type = other RNA; note = bta-miR-11988
aaggggacgacagaggatgaga

SEQ ID NO: 7 - RNA, 22 nt, synthetic construct
  source 1..22: mol_type = other RNA; note = bta-miR-296-3p
gagggttgggcggaggctttcc

SEQ ID NO: 8 - RNA, 24 nt, synthetic construct
  source 1..24: mol_type = other RNA; note = bta-miR-10174-3P
atcacattgccagggattaccacg

SEQ ID NO: 9 - RNA, 21 nt, synthetic construct
  source 1..21: mol_type = other RNA; note = bta-miR-378c
actggacttggagtcagaagt

SEQ ID NO: 10 - RNA, 20 nt, synthetic construct
  source 1..20: mol_type = other RNA; note = bta-miR-27a-3p
ttcacagtggctaagttccg

SEQ ID NO: 11 - RNA, 18 nt, synthetic construct
  source 1..18: mol_type = other RNA; note = novel_118
gggggcggcgggaccggg

SEQ ID NO: 12 - RNA, 20 nt, synthetic construct
  source 1..20: mol_type = other RNA; note = novel_173
catctagactgtgagcttct

SEQ ID NO: 13 - DNA, 23 bp, synthetic construct
  source 1..23: mol_type = other DNA; note = U6 forward primer
cgcttcacgaatttgcgtgtcat

SEQ ID NO: 14 - DNA, 25 bp, synthetic construct
  source 1..25: mol_type = other DNA; note = U6 reverse primer
gcttcggcagcacatatactaaaat

SEQ ID NO: 15 - DNA, 22 bp, synthetic construct
  source 1..22: mol_type = other DNA; note = cel-miR-39-3p forward primer
tcaccgggtgtaaatcagcttg

SEQ ID NO: 16 - DNA, 22 bp, synthetic construct
  source 1..22: mol_type = other DNA; note = bta-miR-2285bn forward primer
aaaacctgaatgaactttttgt

SEQ ID NO: 17 - DNA, 23 bp, synthetic construct
  source 1..23: mol_type = other DNA; note = bta-miR-2285ce forward primer
gaaaacctgaatgaactttttga

SEQ ID NO: 18 - DNA, 21 bp, synthetic construct
  source 1..21: mol_type = other DNA; note = bta-miR-10225b forward primer
tcgagcctgacagatcacaca

SEQ ID NO: 19 - DNA, 20 bp, synthetic construct
  source 1..20: mol_type = other DNA; note = bta-miR-369-5p forward primer
atcgaccgtgttatattcgc

SEQ ID NO: 20 - DNA, 21 bp, synthetic construct
  source 1..21: mol_type = other DNA; note = bta-miR-219-3p forward primer
agaattgtggctggacatctg

SEQ ID NO: 21 - DNA, 22 bp, synthetic construct
  source 1..22: mol_type = other DNA; note = bta-miR-11988 forward primer
aaggggacgacagaggatgaga

SEQ ID NO: 22 - DNA, 22 bp, synthetic construct
  source 1..22: mol_type = other DNA; note = bta-miR-296-3p forward primer
gagggttgggcggaggctttcc

SEQ ID NO: 23 - DNA, 24 bp, synthetic construct
  source 1..24: mol_type = other DNA; note = bta-miR-10174-3p forward primer
atcacattgccagggattaccacg

SEQ ID NO: 24 - DNA, 21 bp, synthetic construct
  source 1..21: mol_type = other DNA; note = bta-miR-378c forward primer
actggacttggagtcagaagt

SEQ ID NO: 25 - DNA, 20 bp, synthetic construct
  source 1..20: mol_type = other DNA; note = bta-miR-27a-3p forward primer
ttcacagtggctaagttccg

SEQ ID NO: 26 - DNA, 18 bp, synthetic construct
  source 1..18: mol_type = other DNA; note = novel_118 forward primer
gcgggaccgggaaaaaaa

SEQ ID NO: 27 - DNA, 21 bp, synthetic construct
  source 1..21: mol_type = other DNA; note = novel_173 forward primer
catctagactgtgagcttcta

SEQ ID NO: 28 - DNA, 22 bp, synthetic construct
  source 1..22: mol_type = other DNA; note = cel-miR-39-3p primer
tcaccgggtgtaaatcagcttg

(19) United States
(12) Patent Application Publication
(10) Pub. No.: US 2024/0392372 A1
(43) Pub. Date: Nov. 28, 2024

(54) Application of Exosome miRNA Biomarkers in Diagnosis and Early Warning of Fatty Liver in Periparturient Dairy Cows

(71) Applicant: Shandong Agricultural University, Taian, Shandong province (CN)

(72) Inventors: Kerong SHI, Taian (CN); Xianpeng HOU, Taian (CN); Tingjun LIU, Taian (CN); Xiaoran YAN, Taian (CN); Sisi FAN, Taian (CN); Xuan ZHANG, Taian (CN)

(21) Appl. No.: 18/671,490

(22) Filed: May 22, 2024

(30) Foreign Application Priority Data
May 23, 2023 (CN) 202310585653.X

(51) Int. Cl. C12Q 1/6883; C12Q 1/6809; C12Q 1/6851

(52) U.S. Cl. CPC C12Q 1/6883; C12Q 1/6809; C12Q 1/6851; C12Q 2600/112; C12Q 2600/178

(57) ABSTRACT

The present application belongs to the technical field of biology. Provided is exosome miRNA biomarkers in diagnosis and early warning of fatty liver in periparturient dairy cows. 12 miRNA biomarkers are discovered in the present application with diagnostic values in recognizing, early warning, identifying, and diagnosing fatty liver disease in dairy cows at corresponding disease stages, especially at least one of three of these miRNAs may be enriched without exosome extraction, and may be used to diagnose, in serum miRNA, whether the dairy cows is suffered from the disease.

### CROSS-REFERENCE TO RELATED APPLICATIONS

The present application claims the benefit of the priority of the Chinese patent application with the application No. 202310585653.X, filed to the China National Intellectual Property Administration on May 23, 2023, the entire content of which is incorporated in this application by reference.

### SEQUENCE LISTING

The instant application contains a Sequence Listing which has submitted electronically in ASCII format and is hereby incorporated by reference in its entirety. Said ASCII copy is named PN243989 SEQ LIST.xml and is 30,687 bytes in size. The sequence listing contains 28 sequences. No new matter is included.

### TECHNICAL FIELD

The present disclosure relates to the technical field of biology, and specifically to an application of exosome miRNA biomarkers in diagnosis and early warning of fatty liver in periparturient dairy cows.

### BACKGROUND OF THE INVENTION

Fatty liver is a highly prevalent and susceptible metabolic disorder in periparturient dairy cows (the period of time between three weeks before and three weeks after a dairy cow gives birth is called a periparturient period). Some studies have shown that, during the periparturient period, 5-10% of the dairy cows have high-grade fatty liver and 30-40% have mid-grade or low-grade fatty liver. The occurrence of the disease reduces milk production and the parturition and reproductive performance of the dairy cows, and results in a higher elimination rate of the dairy cows. Therefore, accurate diagnosis of fatty liver is not only beneficial to the health of the dairy cows and the sustainability of feeding, but also may be adjusted and prevented in advance through early warning diagnosis, so as to reduce the economic loss of farms due to reduced milk production. In addition, the elimination rate of the periparturient dairy cows is reduced, and the economic loss caused by eliminated cows is prevented.

Currently, methods for detecting fatty liver mainly include liver biopsy, serum physiology and biochemistry, proteomics and metabolomics, and a digital image technology. The most accurate and direct method is liver biopsy, but the method requires surgical removal of the liver, causing secondary damage to the dairy cows and increasing losses. A diagnostic method relatively recognized currently in clinical practice is a Y-value method (Reid et al., 1983) proposed by Reid. 3 serum biochemical indexes need to be calculated, but cannot accurately determine the severity of fatty liver disease, such that the method is poor in accuracy, and is not suitable for large-scale ranches (Hu et al., 2018; Zhang et al., 2022). Therefore, there is an urgent need for the industry to develop and research a new diagnostic method that is efficient, accurate, easy to operate and essentially harmless or even non-invasive.

MiRNA (microRNA) is a small non-coding RNA with regulatory functions that is approximately 22 nt in length, which may be produced by almost all cells in the body (Bartel, 2018; Ha et al., 2014). Studies have shown that, miRNAs are involved in almost all developmental and pathological processes in animals (Ha and Kim, 2014). In addition, researchers found that, specific circulating miRNAs exercise the functions of biomarkers for diagnostic discrimination and prognosis of many types of diseases (Jamali et al., 2018).

Exosomes are extracellular vesicles with an endosomal origin with a diameter range of 40-160 nm (Kalluri et al., 2020). The exosomes are widely distributed in various body fluids such as blood, saliva, cerebrospinal fluid, breast milk, and urine, which may carry biomolecules including the miRNAs (Xu et al., 2022). As research further progresses, the exosomes are emerging as a potentially valuable non-invasive liquid biopsy tool for fatty liver progression in periparturient dairy cows.

Compared with other nucleic acids, the miRNAs are more ideal biomarker candidates. The miRNAs are persistent and stable in blood, and the miRNAs may remain relatively stable in the presence of RNA enzymes without being degraded (Mitchell et al., 2008), such that the miRNAs have important values as non-invasive biomarkers in diagnosis (Wang et al., 2009). As the biomarkers, the miRNAs have the advantages of being rapid, sensitive, non-invasive, economic, and efficient, such that the miRNAs become effective biomarkers for the diagnosis and prognosis of many diseases (Jamali et al., 2018). The inventor team of this patent has already found differential miRNAs with the help of human, mouse, and rat fatty liver models in the previous study (CN 105420405 B), partial miRNAs are selected and tested in the serum of cows with suspected fatty liver disease, a group of bovine serum miRNA molecular markers that may be used for predicting fatty liver in dairy cows and metabolic diseases in periparturient dairy cows is finally obtained, and the molecular markers may be used for early detection and diagnosis of diseased cows in dairy cow parturition practices. However, the following problems remain in the aforementioned study:

(1) The miRNA molecular markers screened are not directly derived from cows, and the specificity and sensitivity of the markers need to be improved.

(2) Cow groups are based on changes in several biochemical indicators to determine the probability that a cow is suffered from fatty liver disease, which is far from accurate diagnosis, just a suspected disease, such that diagnostic methods have some limitations and large errors (one study pointed out that, the error in the prevalence of fatty liver in periparturient dairy cows may be as high as about 30-50%, mainly due to temporal and physiological variability in biochemical indicators in the serum, which may lead to difficulties in judgment and misdiagnosis). Correspondingly, the sensitivity and specificity of the bovine serum miRNA molecular markers for diagnosis also need to be further improved.

### SUMMARY OF THE INVENTION

In view of the related art, the present disclosure is intended to provide an application of exosome miRNA biomarkers in diagnosis and early warning of fatty liver in periparturient dairy cows.

In order to implement the above objective, the present disclosure uses the following technical solutions.

A first aspect of the present disclosure provides an application of at least one exosome miRNA in the following 1)-12) in preparation of a kit for diagnosing or early warning fatty liver disease in periparturient dairy cows as a biomarker:
1) bta-miR-2285bn, a nucleotide sequence of which is shown in SEQ ID NO: 1;
2) bta-miR-2285ce, a nucleotide sequence of which is shown in SEQ ID NO: 2;
3) bta-miR-10225b, a nucleotide sequence of which is shown in SEQ ID NO: 3;
4) bta-miR-369-5p, a nucleotide sequence of which is shown in SEQ ID NO: 4;
5) bta-miR-219-3p, a nucleotide sequence of which is shown in SEQ ID NO: 5;
6) bta-miR-11988, a nucleotide sequence of which is shown in SEQ ID NO: 6;
7) bta-miR-296-3p, a nucleotide sequence of which is shown in SEQ ID NO: 7;
8) bta-miR-10174-3p, a nucleotide sequence of which is shown in SEQ ID NO: 8;
9) bta-miR-378c, a nucleotide sequence of which is shown in SEQ ID NO: 9;
10) bta-miR-27a-3p, a nucleotide sequence of which is shown in SEQ ID NO: 10;
11) novel_118, a nucleotide sequence of which is shown in SEQ ID NO: 11; and
12) novel_173, a nucleotide sequence of which is shown in SEQ ID NO: 12.

The 12 exosome miRNAs as the biomarkers all can accurately recognize the fatty liver in the periparturient dairy cows, and an Area Under Curve (AUC) of each exosome miRNA is consistent with diagnostic significance, and has a high clinical diagnostic application value; furthermore, when a plurality of exosome miRNAs are used in combination, the AUC is closer to 1 than a single lipid is used, such that a diagnostic effect is better.

Further, when the plurality of exosome miRNAs are used in combination, on the basis of different exosome miRNA combinations, different diagnostic effects may be realized. Details are as follows.

Mid-grade fatty liver in periparturient dairy cows may be diagnosed with 3 exosome miRNAs of novel_118, bta-miR-2285bn, and bta-miR-2285ce in combination.

High-grade fatty liver in the periparturient dairy cows may be diagnosed with 7 exosome miRNAs of bta-miR-369-5p, bta-miR-219-3p, bta-miR-10225b, novel_173, bta-miR-11988, bta-miR-10174-3p, and bta-miR-378c in combination.

Mid-grade fatty liver and high-grade fatty liver in the periparturient dairy cows may be distinguished with 3 exosome miRNAs of novel_173, bta-miR-378c, and bta-miR-27a-3p in combination.

Periparturient dairy cows with fatty liver and health dairy cows may be distinguished with 6 exosome miRNAs of bta-miR-369-5p, novel_118, bta-miR-10225b, bta-miR-219-3p, bta-miR-296-3p, and bta-miR-10174-3p in combination.

A second aspect of the present disclosure provides an application of a reagent for testing an exosome miRNA in preparation of products for non-invasive recognition of fatty liver disease in periparturient dairy cows.

The exosome miRNA is at least one miRNA shown in SEQ ID NO: 1-SEQ ID NO: 12.

Further, the reagent is a reagent for testing miRNAs in serum exosomes of dairy cows, or a reagent for directly testing miRNAs in the serum.

Preferably, the reagent contains a primer for qPCR detection, with a sequence as shown in SEQ ID NO: 16-SEQ ID NO: 27.

The present disclosure has the following beneficial effects.

(1) 12 miRNA biomarkers discovered in the present disclosure with diagnostic values in recognizing, early warning, identifying, and diagnosing fatty liver disease in dairy cows at corresponding disease stages, especially at least one of three of these miRNAs may be enriched without exosome extraction, and may be used to diagnose, in serum miRNA, whether the dairy cows is suffered from the disease. The biomarkers of the present disclosure are used for identifying and diagnosing the dairy cows with fatty liver, and have high specificity and sensitivity; the markers are stable in nature, simple in operation, low in price, and are a non-invasive detection means; and the markers have no harm or injury to dairy cows, which are in line with the concept of animal welfare and healthy farming, such that the biomarkers may be widely applied to large-scale farming of the dairy cows in the future, and healthy and efficient development of the dairy industry is promoted.

(2) In conjunction with the screening and verification of 43 periparturient dairy cows, it found that the miRNA biomarkers provided in the present disclosure have high sensitivity and specificity on recognition and diagnosis of the fatty liver, and has significant use value and significance for future diagnosis of the fatty liver in the periparturient dairy cows.

(3) The miRNA biomarkers screened in the present disclosure are of bovine origin, and are more targeted; in the present disclosure, the health status of dairy cows is determined after accurate diagnosis by means of liver biopsy, which may completely diagnose the health or disease severities of cows; based on the above improvement, the miRNA biomarkers discovered in the present disclosure is more sensitive and stronger in specificity than miRNA biomarkers (bovine serum microRNA molecular markers for fatty liver disease in dairy cow and metabolic disorders related to the periparturient dairy cow, ZL201610034351.3) discovered by the team.

### BRIEF DESCRIPTION OF DRAWINGS

FIG. 1 is a discovery flowchart of a biomarker according to the present disclosure.

FIG. 2 shows fat content of liver tissue of each group of a sequenced cow population.

FIGS. 3A, 3B, and 3C are a volcano plot of differential miRNAs between groups. A screening standard is |log 2 (FC)|≥0.5; P≤0.05.

FIG. 4 is a Venn diagram of differential miRNAs between groups. According to a volcano plot screening result, a total of 28 differentially-expressed miRNAs (duplicated miRNAs removed) are obtained.

FIG. 5 shows results of a Wayne diagram of a total of 12 miRNAs (duplicated miRNAs removed) between groups obtained by Receiver Operating Characteristic (ROC) curve screening analysis. A screening standard is AUC>0.7 (simultaneously P<0.05). In addition, an SFL vs MFL screening standard is AUC>0.7.

FIG. 6 shows ROC curves of 3 miRNAs screened by an ROC curve of a MFL vs Norm group and ROC curves for combined diagnosis of the 3 miRNAs of the group. A screening standard is AUC>0.7, P<0.05.

FIG. 7 shows ROC curves of 7 miRNAs screened by an ROC curve of a SFL vs Norm group and ROC curves for combined diagnosis of the 7 miRNAs of the group. A screening standard is AUC>0.7, P<0.05.

FIG. 8 shows ROC curves of 3 miRNAs screened by an ROC curve of a SFL vs MFL group and ROC curves for combined diagnosis of the 3 miRNAs of the group. A screening standard is AUC>0.7.

FIG. 9 shows ROC curves of 6 miRNAs screened by an ROC curve of a FL vs Norm group and ROC curves for combined diagnosis of the 6 miRNAs of the group. A screening standard is AUC>0.7, P<0.05.

FIG. 10 is a sequencing expression violin plot of 12 miRNAs obtained through screening.

FIG. 11 shows verification of fat content of liver tissue of each group of a new cow population. A total of 12 dairy cows with health status determined through liver biopsy diagnosis in three groups in a new population are verified.

FIGS. 12A, 12B, and 12C show verification of 3 miRNAs that still have diagnostic values in serum miRNAs. Vertical coordinates, red broken lines, rounded black dots, and bar charts on the left side of the top three images in the figure represent the expression of the miRNA in a serum verification set; and vertical coordinates, blue broken lines, and square blue dots on the right side in the figure represent the expression of the miRNA in a sequencing set.

### DETAILED DESCRIPTION OF THE INVENTION

It should be noted that, the following detailed description is exemplary and intended to provide further description of the present application. Unless otherwise defined, all technical and scientific terms used herein have the same meaning as commonly understood by those of ordinary skill in the art to which the present application belongs.

As described above, due to errors in the source of the miRNAs and methods for determining whether dairy cows are suffered from fatty liver, the specificity and accuracy of bovine serum miRNAs developed by an inventor team in previous studies to diagnose fatty liver in the dairy cows still need to be further improved.

In view of this, further research is performed in the present disclosure. Serum exosomes contain high levels of miRNAs. The exosomes are enriched first, and then more miRNAs may be enriched and incorporated in a database for subsequent screening. Therefore, in the present disclosure, starting with serum exosome miRNAs, the exosome miRNAs for diagnosing or early warning fatty liver disease in periparturient dairy cows can be screened.

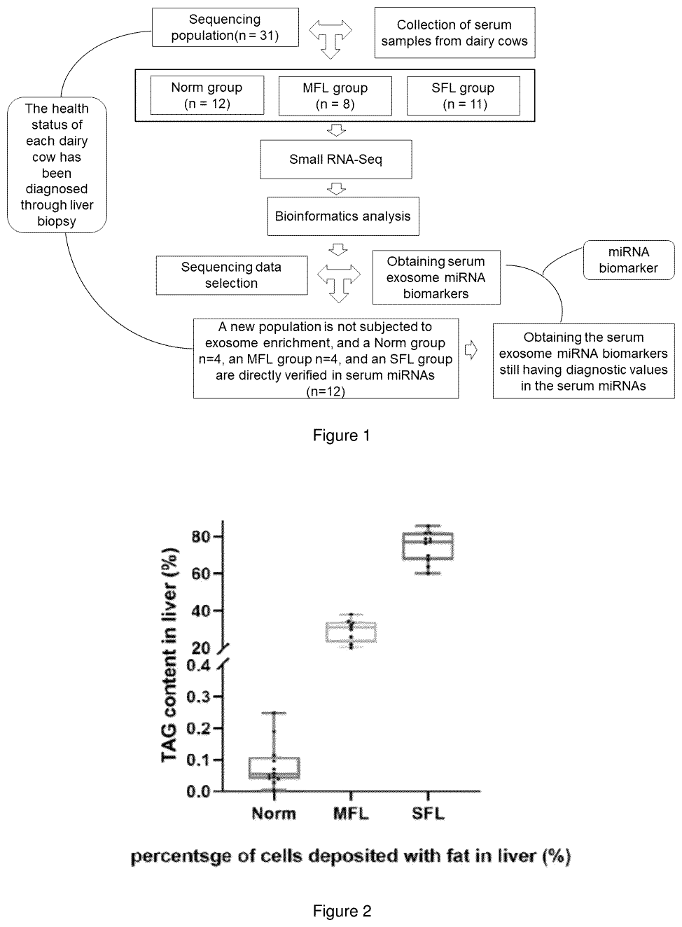

A process of discovering exosome RNA biomarkers in the present disclosure is shown in FIG. 1. Details are as follows.

A periparturient dairy cow population that has been subjected to liver biopsy identification is divided into a sequencing set (n=31) and a serum verification set (n=12). The sequencing set (n=31) includes a normal group (Norm, n=12), a mid-grade fatty liver group (MFL, n=8), and a high-grade fatty liver group (SFL, n=11). Screening of serum exosome miRNA biomarkers is performed in the serum of dairy cows in the sequencing set, and Small RNA-seq is performed on the biomarkers first, so as to obtain a full miRNA expression profile.

Further, a multivariate statistical Orthogonal Partial Least Squares Discriminant Analysis (OPLS-DA) model is used, and on the basis of a nonlinear iterative partial least squares algorithm, dimension reduction processing (supervised classification model) may also be performed. A relationship model between miRNA expression and sample categories that is required for sequencing may be established with orthogonal partial least squares regression, such that the difference between classification groups may be reflected to the greatest extent, so as to achieve modeling prediction of the sample categories, and Variable Importance in Projection (VIP) value of each miRNA is calculated as subsequent reference.

Further, differential miRNAs between groups are screened, according to different groups, from the miRNAs obtained through sequencing. Statistical analysis is performed on all the miRNAs that are expressed in comparison groups MFL vs Norm, SFL vs Norm, and SFL vs MFL, and analysis is performed with DESeq2 based on negative binomial distribution (Love et al., 2014). A screening standard of differential genes is |log 2 (FC)|≥0.5; P≤0.05, and results between groups are shown in a differential volcano plot.

Further, for the obtained miRNAs differentially expressed among the groups, the diagnostic values of the miRNAs obtained are evaluated by means of ROC curve screening analysis. When the AUC of an ROC curve is between 0.7 and 0.9 (also P<0.05), it indicates that the AUC is moderately accurate. When the AUC of the ROC curve is greater than 0.9 (also P<0.05), it indicates that the AUC is highly accurate. The miRNAs with AUC>0.7 (also P<0.05) are selected between the groups; the miRNAs with more than moderate diagnostic capabilities are obtained through screening; and the obtained miRNAs are used as candidate miRNA biomarkers.

Further, the MFL group and the SFL group are used as disease groups, which are recorded as FL groups; and the diagnostic values of the miRNAs are evaluated by performing ROC curve screening analysis. The screening standard is also AUC>0.9, P<0.05.

At this time, the obtained miRNAs in each group are serum exosome miRNAs from the periparturient dairy cows with more than moderate diagnostic capabilities for fatty liver, and results of performing combined diagnosis on the miRNAs between the groups show that a higher diagnostic application value is realized through combination. Since there are fewer biomarkers obtained in SFL vs MFL, a screening range is enlarged, and the screening standard is AUC>0.7.

Further, combined diagnosis analysis is performed on each group of the candidate miRNAs obtained through ROC curve screening analysis; and combined diagnosis analysis is performed by means of Logistic regression analysis and ROC curve analysis, so as to obtain an AUC value (AUC>0.7) analyzed through combined diagnosis, a P value (P<0.05), and a combined diagnosis ROC curve.

Further, since the conditions for exosome extraction are relatively demanding, as evidenced by the cumbersome and time-consuming operations, in the present disclosure, when the serum verification set is used to perform verification, exosome enrichment is not performed, but the miRNAs are directly extracted from the serum of the periparturient dairy cows that have been subjected to liver biopsy to diagnose health status.

Therefore, in the serum verification set (n=12) (the health status of the dairy cows has been diagnosed through liver biopsy), the Norm group (normal group, n=4), the MFL group (mid-grade fatty liver group, n=4), and the SFL group (high-grade fatty liver group, n=4), exosome extraction and enrichment is not performed in the serum verification set, but RT-qPCR verification and ROC curve diagnosis analysis verification are directly performed, in the serum miRNAs, on the candidate exosome miRNA markers that screened through sequencing.

A combination of an internal reference and an external reference is used during verification; U6 is used as the internal reference, and a cel-miR-39-3p external reference standard that is not present in dairy cows is selected as the external reference. Through comparison and considering that the addition of the external reference increases the burden of verification during specific usage, in the present disclosure, the internal reference U6 may be directly used for subsequent quantification during specific usage.

RT-qPCR verification and ROC curve diagnosis analysis verification are performed on the obtained miRNAs with a tailing method, so as to analyze and verify whether the candidate serum exosome miRNA biomarkers still have diagnostic capabilities and values without exosome enrichment in the serum miRNAs.

In the present disclosure, 12 serum exosome miRNA biomarkers with diagnostic values are finally discovered, and the 12 serum exosome miRNA biomarkers respectively are: bta-miR-369-5p, bta-miR-219-3p, bta-miR-10225b, bta-miR-10174-3p, bta-miR-2285bn, bta-miR-2285ce, bta-miR-11988, bta-miR-378c, bta-miR-296-3p, bta-miR-27a-3p, novel_118, and novel_173. At least one of the 12 serum exosome miRNA biomarkers may be used as a non-invasive serum exosome miRNA biomarker for recognizing dairy cows with fatty liver disease at corresponding disease severities; and 3 of the miRNAs are discovered, and the miRNA is at least one of bta-miR-369-5p, bta-miR-219-3p, and bta-miR-10174-3p that may not be enriched through exosome extraction, and the recognition, early warning, or diagnosis of the fatty liver in the periparturient dairy cows may be directly performed in the serum miRNA.

Sequence information of the 12 serum exosome miRNAs with diagnostic values in the present disclosure is shown in Table 1 and SEQ ID NO: 1-SEQ ID NO: 12 in the sequence listing.

TABLE 1
miRNA mature sequence and miR Base
database accession number

| miR Base | | |
|---|---|---|
| database | | Sequence of |
| accession | | mature miRNAs |
| number | miRNA | (direction 5′ to 3′) |
| MIMAT0046625 | bta-miR- | AAAACCUGAAUGAACUUUUUGU |
| | 2285bn | (SEQ ID NO: 1) |
| MIMAT0046657 | bta-miR- | GAAAACCUGAAUGAACUUUUUG |
| | 2285ce | A (SEQ ID NO: 2) |
| MIMAT0041127 | bta-miR- | UCGAGCCUGACAGAUCACACA |
| | 10225b | (SEQ ID NO: 3) |
| MIMAT0003801 | bta-miR- | AUCGACCGUGUUAUAUUCGC |
| | 369-5p | (SEQ ID NO: 4) |
| MIMAT0012547 | bta-miR- | AGAAUUGUGGCUGGACAUCUG |
| | 219-3p | (SEQ ID NO: 5) |
| MIMAT0046393 | bta-miR- | AAGGGGACGACAGAGGAUGAGA |
| | 11988 | (SEQ ID NO: 6) |
| MIMAT0009273 | bta-miR- | GAGGGUUGGGCGGAGGCUUUCC |
| | 296-3p | (SEQ ID NO: 7) |
| MIMAT0040928 | bta-miR- | AUCACAUUGCCAGGGAUUACCA |
| | 10174-3p | CG (SEQ ID NO: 8) |
| MIMAT0025551 | bta-miR- | ACUGGACUUGGAGUCAGAAGU |
| | 378c | (SEQ ID NO: 9) |
| MIMAT0003532 | bta-miR- | UUCACAGUGGCUAAGUUCCG |
| | 27a-3p | (SEQ ID NO: 10) |
| — | novel_118 | gggggcggcgggaccggg |
| | | (SEQ ID NO: 11) |
| — | novel_173 | caucuagacugugagcuucu |
| | | (SEQ ID NO: 12) |

Note:
according to the WIPO ST.26 standard, uracil “U” in the miRNA sequences in the table is represented by “T” in the sequence listing.

In order to enable those skilled in the art to understand the technical solutions of the present application more clearly, the technical solutions of the present application will be described in detail with reference to specific embodiments.

Unspecified test materials used in the embodiments of the present disclosure are conventional test materials in the art and are commercially available.

### Embodiment 1: Biological Sample Situations and Methods Involved in the Present Disclosure

Chinese periparturient Holstein cows were used as biological samples, and the biological samples were divided into a sequencing set (n=31) and a serum verification set (n=12).

According to results of fat content of the collected liver tissue, the degree of fatty liver in dairy cows was classified (referring to FIG. 2 for diagnosis situations); and according to different fat content of diseased dairy cows, all sequencing samples were divided into three groups (n=31): the fat content of a normal group (Norm group) was 0.082%±0.071% (n=12); the fat content of a mid-grade fatty liver group (MFL group) was 29.49%±6.30% (n=8); and the fat content of a high-grade fatty liver group (SFL group) was 74.70%±8.23% (n=11). Then exosome extraction, exosome total RNA extraction, and small RNA sequencing were performed.

### Embodiment 2: Preliminary Screening Through Differential Expression Analysis

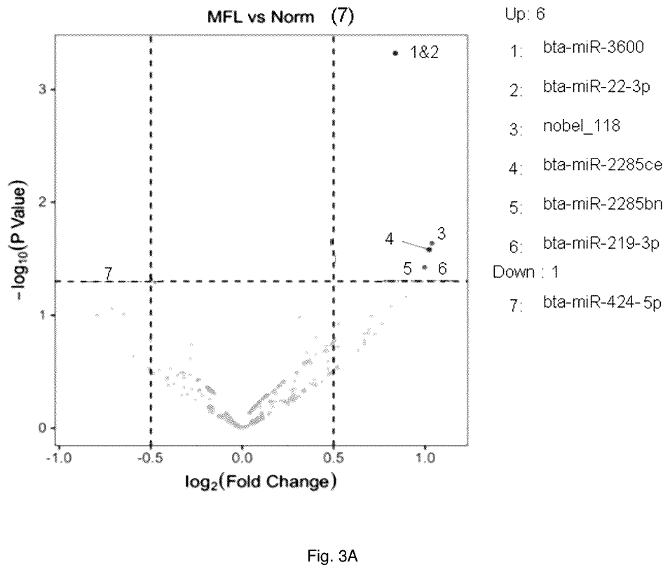

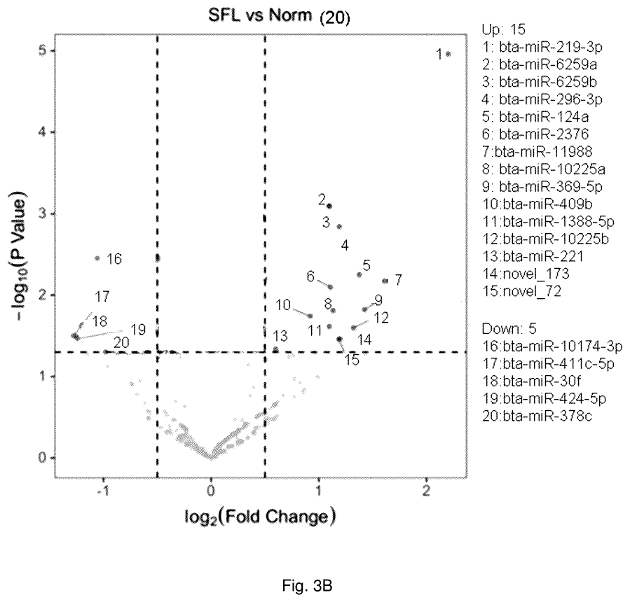

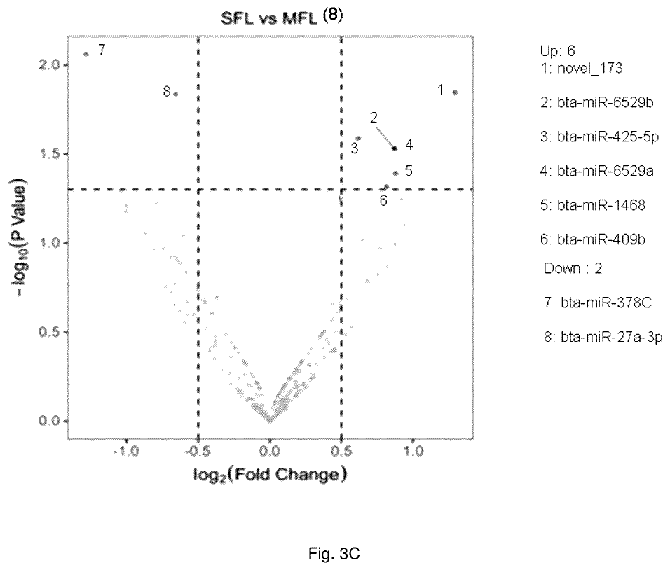

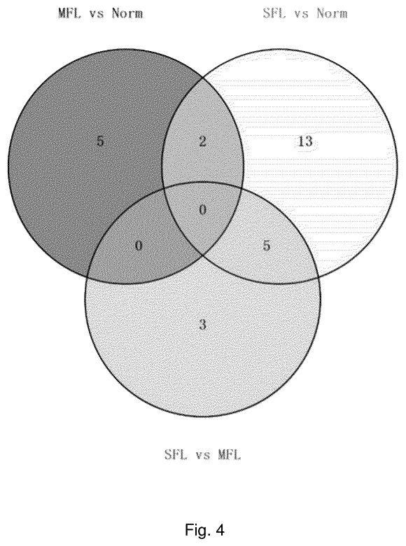

A total of 309 miRNAs were obtained through small RNA sequencing in Embodiment 1. Through DESeq2 analysis, according to a screening standard (as shown in FIGS. 3A-3C) of |log 2 (FC)|≥0.5, P≤0.05, three groups were obtained, which were a MFL vs Norm comparison group (7, as shown in FIG. 3A), where the obtained miRNA was bta-miR-22-3p, bta-miR-3600, novel_118, bta-miR-2285bn, bta-miR-2285ce, bta-miR-219-3p, or bta-miR-424-5p; a SFL vs Norm comparison group (20, as shown in FIG. 3B), where the obtained miRNA was bta-miR-219-3p, bta-miR-424-5p, bta-miR-296-3p, bta-miR-10174-3p, bta-miR-124a, bta-miR-11988, bta-miR-2376, bta-miR-369-5p, bta-miR-10225a, bta-miR-1388-5p, bta-miR-10225b, bta-miR-411c-5p, bta-miR-30f, novel_72, bta-miR-221, bta-miR-6529a, bta-miR-6529b, bta-miR-409b, novel_173, or bta-miR-378c; and a SFL vs MFL comparison group (8, as shown in FIG. 3C), where the obtained miRNA was bta-miR-6529a, bta-miR-6529b, bta-miR-409b, novel_173, bta-miR-378c, bta-miR-27a-3p, bta-miR-425-5p, and bta-miR-1468, duplicated miRNAs were removed, with a total of 28 miRNAs significantly differentially expressed (as shown in FIG. 4).

### Embodiment 3: miRNA Biomarkers Obtained Through ROC Curve Screening

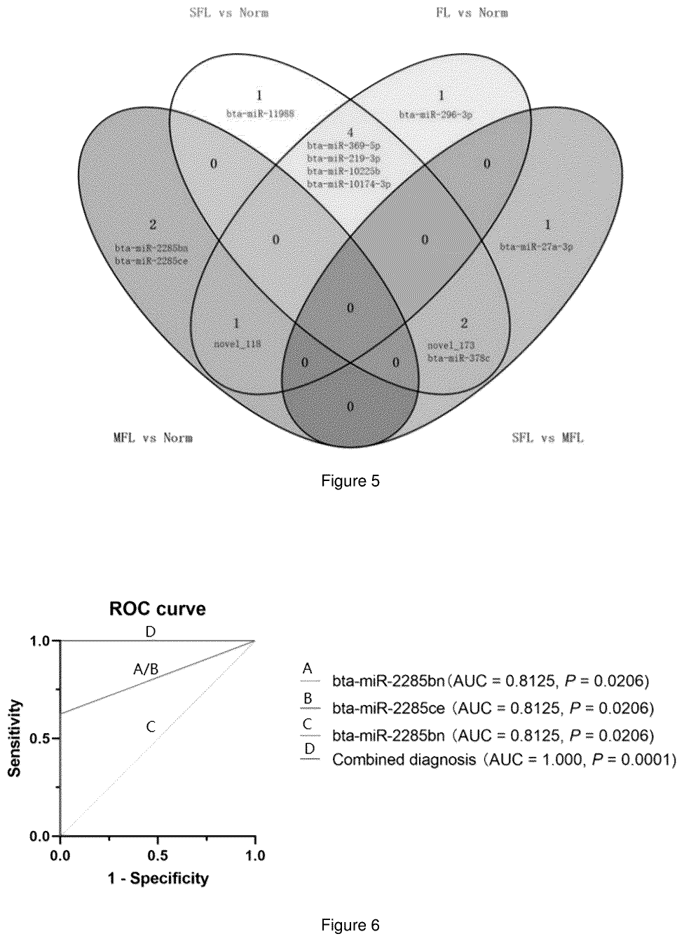

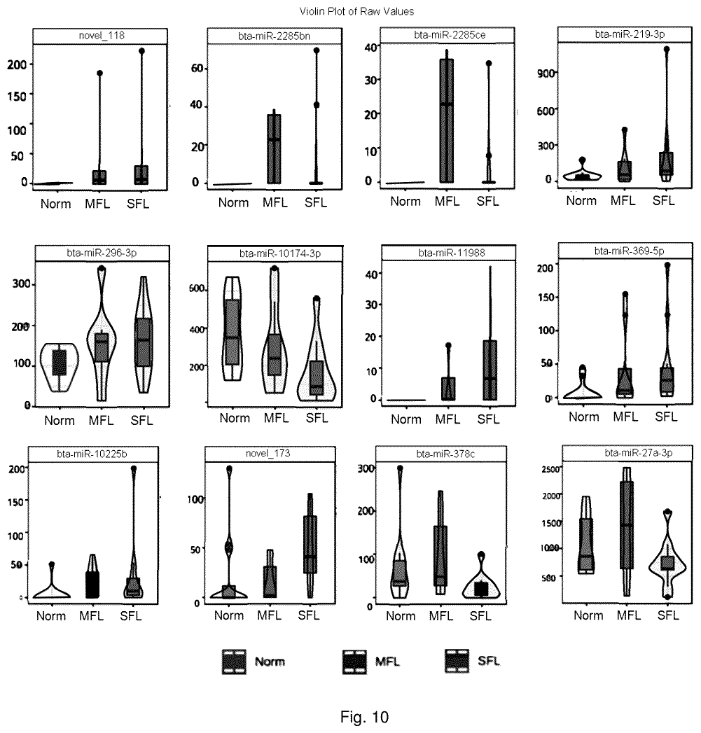

Diagnostic capability ROC screening analysis was further performed on 28 miRNAs significantly differentially expressed that were preliminarily screened in Embodiment 2; the miRNAs with AUC>0.7 and P<0.05 were selected, so as to obtain MFL vs Norm (3) (as shown in Table 2), SFL vs Norm (7) (as shown in Table 3), SFL vs MFL (3) (as shown in Table 4), and FL vs Norm (6) (as shown in Table 5), duplicated miRNAs were removed, with a total of 12 serum exosome miRNA biomarkers with diagnostic significance in periparturient dairy cows (as shown in FIG. 5): bta-miR-2285bn, bta-miR-2285ce, novel_118, bta-miR-11988, bta-miR-369-5p, bta-miR-219-3p, bta-miR-10225b, bta-miR-10174-3p, novel_173, bta-miR-378c, bta-miR-296-3p, bta-miR-27a-3p. A sequencing expression violin plot of the 12 miRNAs was shown in FIG. 10.

TABLE 2
MFL vs Norm diagnosis of mid-grade ROC analysis
results (3 miRNAs + combined diagnosis analysis)

| | | | | 95% | | | | Changes |
|---|---|---|---|---|---|---|---|---|
| | | | | Confi- | | | | (sequencing |
| | | | | dence | | | Best | set) log2 |
| | | | P | Interval | | | cutoff | (FC) |
| MiRNA | AUC | SEM(±) | value | (CI) | Sensitivity | Specificity | value | (Seq) |
| novel_118 | 0.813 | 0.113 | 0.021 | 0.591-1.00 | 0.625 | 1.000 | 0.625 | 1.038 |
| bta-miR- | 0.813 | 0.113 | 0.021 | 0.591-1.00 | 0.625 | 1.000 | 0.625 | 1.023 |
| 2285bn | | | | | | | | |
| bta-miR- | 0.813 | 0.113 | 0.021 | 0.591-1.00 | 0.625 | 1.000 | 0.625 | 1.023 |
| 2285ce | | | | | | | | |
| Combined | 1.000 | 0.000 | 0.0001 | 1.000 | 1.000 | 1.000 | 1.000 | — |
| diagnosis | | | | | | | | |

TABLE 3
SFL vs Norm diagnosis of high-grade ROC analysis results (7 miRNAs)

| | | | | | | | | Changes |
|---|---|---|---|---|---|---|---|---|
| | | | | | | | | (sequencing |
| | | | | | | | | set) |
| | | | | | | | Best | log2 |
| | | | P | 95% | | | cutoff | (FC) |
| MiRNA | AUC | SEM(±) | value | CI | Sensitivity | Specificity | value | (Seq) |
| bta-miR- | 0.894 | 0.074 | 0.001 | 0.748- | 1.000 | 0.833 | 0.833 | 1.423 |
| 369-5p | | | | 1.000 | | | | |
| bta-miR- | 0.871 | 0.077 | 0.003 | 0.720- | 0.909 | 0.75 | 0.659 | 2.199 |
| 219-3p | | | | 1.000 | | | | |
| bta-miR- | 0.807 | 0.099 | 0.013 | 0.613- | 0.727 | 0.917 | 0.644 | 1.320 |
| 10225b | | | | 1.000 | | | | |
| novel_ | 0.792 | 0.103 | 0.018 | 0.591- | 0.909 | 0.75 | 0.659 | 1.195 |
| 173 | | | | 0.993 | | | | |
| bta-miR- | 0.773 | 0.105 | 0.027 | 0.568- | 0.545 | 1.000 | 0.545 | 1.608 |
| 11988 | | | | 0.978 | | | | |
| bta-miR- | 0.833 | 0.086 | 0.007 | 0.664- | 0.636 | 0.917 | 0.553 | −1.060 |
| 10174-3p | | | | 1.000 | | | | |
| bta-miR- | 0.705 | 0.114 | 0.097 | 0.482- | 0.636 | 0.833 | 0.47 | −0.982 |
| 378c | | | | 0.927 | | | | |
| Combined | 1.000 | 0.000 | <0.0001 | 1.000 | 1.000 | 1.000 | 1.000 | — |
| diagnosis | | | | | | | | |

TABLE 4
High-grade ROC analysis results (3 miRNAs) of diagnosis
in SFL vs MFL self-diseased individuals

| | | | | | | | | Changes |
|---|---|---|---|---|---|---|---|---|
| | | | | | | | | (sequencing |
| | | | | | | | Best | set) |
| | | | P | 95% | | | cutoff | log2 (FC) |
| MiRNA | AUC | SEM(±) | value | CI | Sensitivity | Specificity | value | (Seq) |
| novel_ | 0.795 | 0.106 | 0.032 | 0.588- | 0.909 | 0.625 | 0.534 | 1.292 |
| 173 | | | | 1.000 | | | | |
| bta-miR- | 0.739 | 0.118 | 0.083 | 0.507- | 0.909 | 0.5 | 0.409 | −1.284 |
| 378c | | | | 0.970 | | | | |
| bta-miR- | 0.727 | 0.133 | 0.099 | 0.467- | 0.909 | 0.625 | 0.534 | −0.657 |
| 27a-3p | | | | 0.988 | | | | |
| Combined | 0.921 | 0.065 | 0.002 | 0.793- | 1.000 | 0.750 | 0.750 | — |
| diagnosis | | | | 1.000 | | | | |

TABLE 5
FL vs Norm diagnosis of disease ROC analysis results (6 miRNAs)

| | | | | | | | | Changes |
|---|---|---|---|---|---|---|---|---|
| | | | | | | | | (sequencing |
| | | | | | | | Best | set) |
| | | | P | 95% | | | cutoff | log2 (FC) |
| MiRNA | AUC | SEM(±) | value | CI | Sensitivity | Specificity | value | (Seq) |
| bta-miR- | 0.842 | 0.081 | 0.002 | 0.683- | 0.895 | 0.833 | 0.728 | 2.768 |
| 369-5p | | | | 1.000 | | | | |
| novel_ | 0.816 | 0.076 | 0.004 | 0.666- | 0.632 | 1.000 | 0.632 | 2.686 |
| 118 | | | | 0.966 | | | | |
| bta-miR- | 0.809 | 0.085 | 0.004 | 0.643- | 0.737 | 0.917 | 0.654 | 1.502 |
| 10225b | | | | 0.975 | | | | |
| bta-miR- | 0.789 | 0.083 | 0.007 | 0.627- | 0.737 | 0.833 | 0.57 | 2.259 |
| 219-3p | | | | 0.952 | | | | |
| bta-miR- | 0.719 | 0.092 | 0.043 | 0.539- | 0.526 | 1.000 | 0.526 | 0.605 |
| 296-3p | | | | 0.899 | | | | |
| bta-miR- | 0.746 | 0.088 | 0.023 | 0.573- | 0.842 | 0.583 | 0.425 | −0.809 |
| 10174-3p | | | | 0.918 | | | | |
| Combined | 1.000 | 0.000 | <0.0001 | 1.000 | 1.000 | 1.000 | 1.000 | — |
| diagnosis | | | | | | | | |

### Embodiment 4: Combined Diagnosis Analysis Respectively Performed on miRNA Biomarkers Obtained in Each Group

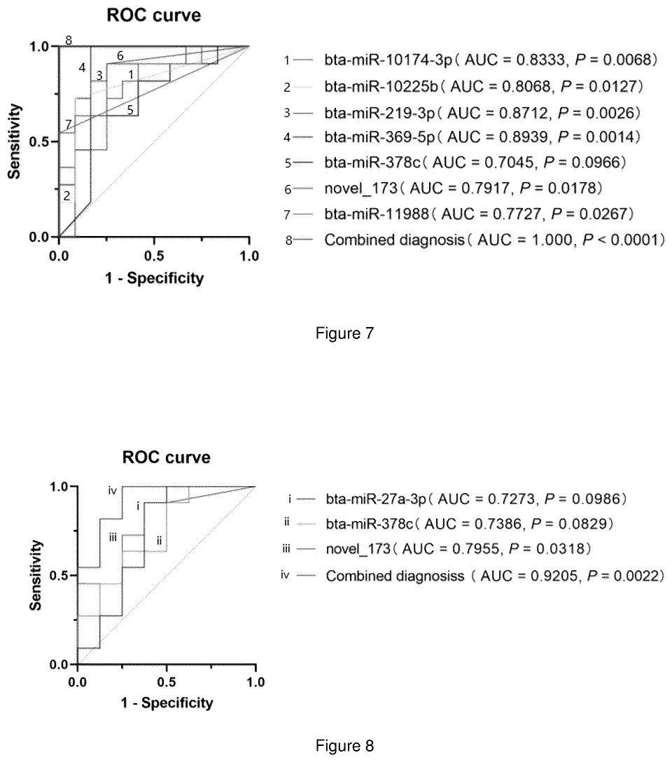

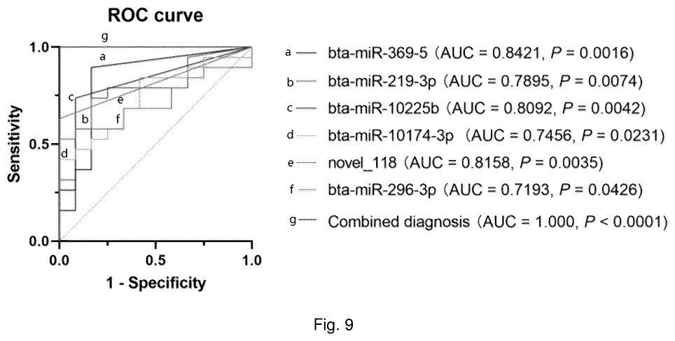

In order to improve the diagnostic efficiency of the miRNA biomarkers obtained in Embodiment 3, combined diagnosis analysis was respectively performed on the miRNA biomarkers obtained in each group, as shown in FIG. 6 and Table 2, the combined diagnosis AUC=1 (P=0.0001) of 3 miRNAs in the MFL vs Norm comparison group had a high diagnostic effect; as shown in FIG. 7 and Table 3, the combined diagnosis AUC=1 (P<0.0001) of 7 miRNAs in the SFL vs Norm comparison group had a high diagnostic effect; as shown in FIG. 8 and Table 4, the combined diagnosis AUC=0.921 (P=0.002) of 3 miRNAs in the SFL vs MFL comparison group also had a high diagnostic effect; and as shown in FIG. 9 and Table 5, the combined diagnosis AUC=1 (P<0.0001) of 6 miRNAs in the FL vs Norm comparison group had a high diagnostic effect.

Therefore, on the basis of different combinations of exosome miRNA biomarkers, different diagnostic effects might be realized. Details were as follows.

Mid-grade fatty liver in periparturient dairy cows might be diagnosed with 3 exosome miRNAs of novel_118, bta-miR-2285bn, and bta-miR-2285ce in combination (referring to Table 2).

High-grade fatty liver in the periparturient dairy cows might be diagnosed with 7 exosome miRNAs of bta-miR-369-5p, bta-miR-219-3p, bta-miR-10225b, novel_173, bta-miR-11988, bta-miR-10174-3p, and bta-miR-378c in combination (referring to Table 3).

Mid-grade fatty liver and high-grade fatty liver in the periparturient dairy cows might be distinguished with 3 exosome miRNAs of novel_173, bta-miR-378c, and bta-miR-27a-3p in combination (referring to Table 4).

Considering that in an actual production process, the diagnosis of fatty liver should mainly address the issue of firstly distinguishing whether the dairy cows fed were suffered from the fatty liver (regardless of disease severities), and secondly distinguishing the disease severities of the dairy cows, so as to provide appropriate treatments, thereby reducing and saving medical and manpower resources, 6 exosome miRNAs of bta-miR-369-5p, novel_118, bta-miR-10225b, bta-miR-219-3p, bta-miR-296-3p, and bta-miR-10174-3p were used in combination (referring to Table 5), such that periparturient dairy cows with fatty liver and health dairy cows might be distinguished.

A method for diagnosing the conditions of the periparturient dairy cows using the exosome miRNA biomarkers of the present disclosure included the following operation.

Blood samples of a healthy cow and a suspected cow or a cow to be diagnosed were collected; the serum was separated, and serum miRNAs (which might also be the miRNAs without exosome enrichment) were extracted; a relative expression for marking the miRNAs was detected by means of real-time fluorescence quantitative PCR; the relative expression was compared with an expression level of the healthy cow, the trend of changes in the fatty liver was the same, it indicated that there was a hidden danger in the suspected cow.

Criteria for determination: the expression level was increased when log 2 (FC)>0 and was decreased when log 2 (FC)<0.

When bta-miR-2285bn or bta-miR-2285ce in Table 2 was used as a diagnostic marker, the cow to be detected showed an increased expression compared to the healthy cow, it indicated that there was a likelihood that the cow to be detected was suffered from mid-grade fatty liver. Otherwise, if bta-miR-10174-3p (as shown in Table 3) was used as the diagnostic marker, and the serum of the cow to be detected showed a reduced expression level compared to the healthy cow, it indicated that there was a likelihood that the cow to be detected was suffered from high-grade fatty liver.

### Embodiment 5: Direct Verification of miRNA Biomarkers in Serum miRNA without Exosome Extraction and Enrichment

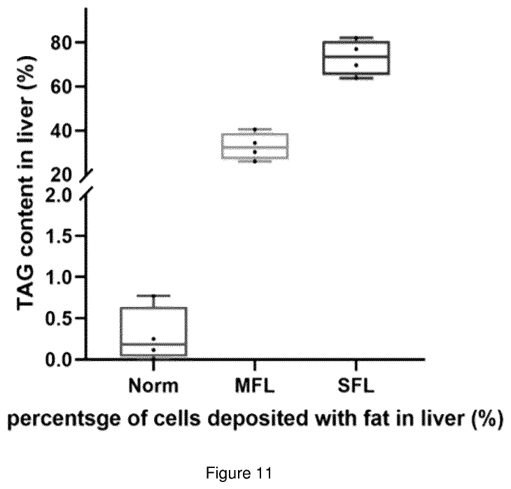

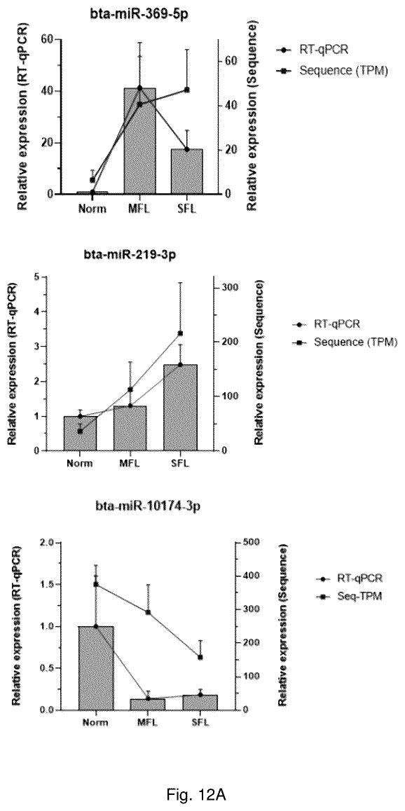

In the serum verification set (n=12) (as shown in FIG. 11), the fat content of the Norm group (normal group, n=4) was 0.28%±0.34%, the fat content of the MFL group (mid-grade fatty liver group, n=4) was 32.75%±6.21%, and the fat content of the SFL group (high-grade fatty liver group, n=4) was 73.09%±8.03%. For ease of application, and to reduce operation steps, exosome extraction and enrichment was not performed on a new population, but RT-qPCR verification and ROC curve diagnosis analysis verification were directly performed, in the serum, on the exosome miRNA markers that were screened from the sequencing set.

Further, a combination of an internal reference and an external reference was used during verification; the internal reference used U6 (a primer sequence was shown in Table 6, and SEQ ID NOs: 13-14 in the sequence table), and the external reference selected a cel-miR-39-3p external reference standard (a primer sequence was shown in Table 6, and SEQ ID NO: 15 in the sequence table) that was not present in dairy cows. Through comparison, and considering that the addition of the external reference increased the burden of verification during specific usage, in the present disclosure, the internal reference U6 might be directly used for subsequent quantification during specific usage; the miRNAs to be detected were verified with a poly-A tailing reverse transcription method; detection was performed with a “miRcute enhanced miRNA fluorescence quantitative detection kit (SYBR Green)(FP411)”; and a quantitative primer sequence used for detection of each miRNA biomarker was shown in Table 7 and SEQ ID NOs: 16-27 in sequence table.

The sequence (5′-3′) of cel-miR-39-3p(MIMAT0000010) was UCACCGGGUGUAAAUCAGCUUG. (SEQ ID NO: 28)

Note: according to a WIPO ST.26 standard, uracil “U” in the miRNA sequence was represented by “T” in the sequence listing.

TABLE 6
Internal and external references and
primers thereof used during verification

| Name | RT primer (5′ to 3′) |
|---|---|
| cel-miR-39-3p | TCACCGGGTGTAAATCAGCTTG |
| (Forward primer) | (SEQ ID NO: 15) |
| U6 (Forward | CGCTTCACGAATTTGCGTGTCAT |
| primer) | (SEQ ID NO: 13) |
| U6 (Reverse | GCTTCGGCAGCACATATACTAAAAT |
| primer) | (SEQ ID NO: 14) |

TABLE 7
miRNA forward primer sequence

| Accession | | |
|---|---|---|
| number | Name | RT primer (5′ to 3′) |
| MIMAT0046625 | bta-miR- | AAAACCTGAATGAACTTTTTGT |
| | 2285bn | (SEQ ID NO: 16) |
| MIMAT0046657 | bta-miR- | GAAAACCTGAATGAACTTTTTG |
| | 2285ce | A (SEQ ID NO: 17) |
| MIMAT0041127 | bta-miR- | TCGAGCCTGACAGATCACACA |
| | 10225b | (SEQ ID NO: 18) |
| MIMAT0003801 | bta-miR- | ATCGACCGTGTTATATTCGC |
| | 369-5p | (SEQ ID NO: 19) |
| MIMAT0012547 | bta-miR- | AGAATTGTGGCTGGACATCTG |
| | 219-3p | (SEQ ID NO: 20) |
| MIMAT0046393 | bta-miR- | AAGGGGACGACAGAGGATGAGA |
| | 11988 | (SEQ ID NO: 21) |
| MIMAT0009273 | bta-miR- | GAGGGTTGGGCGGAGGCTTTCC |
| | 296-3p | (SEQ ID NO: 22) |
| MIMAT0040928 | bta-miR- | ATCACATTGCCAGGGATTACCA |
| | 10174-3p | CG (SEQ ID NO: 23) |
| MIMAT0025551 | bta-miR- | ACTGGACTTGGAGTCAGAAGT |
| | 378c | (SEQ ID NO: 24) |
| MIMAT0003532 | bta-miR- | TTCACAGTGGCTAAGTTCCG |
| | 27a-3p | (SEQ ID NO: 25) |
| — | novel_118 | GCGGGACCGGGAAAAAAA |
| | | (SEQ ID NO: 26) |
| — | novel_173 | CATCTAGACTGTGAGCTTCTA |
| | | (SEQ ID NO: 27) |

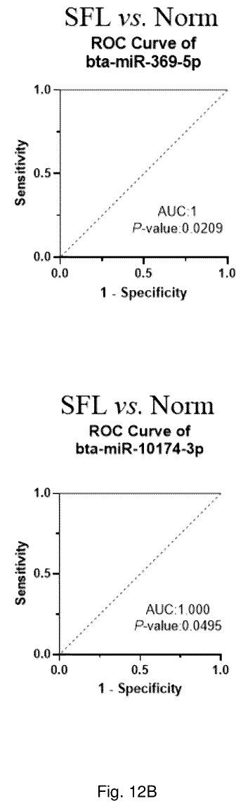

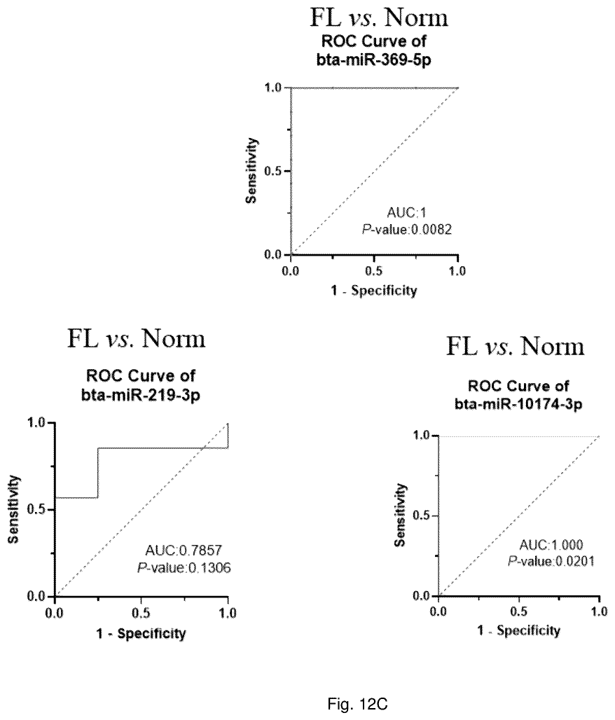

Candidate miRNAs with AUC>0.7, P<0.05 were selected, so as to obtain a SFL vs MFL (3) (referring to Table 8 and FIGS. 12A and 12B): bta-miR-369-5p (AUC=1, P=0.0209), bta-miR-219-3p (AUC=0.9375, P=0.0433), and bta-miR-10174-3p (AUC=1, P=0.0495); and a FL vs Norm (2) (referring to Table 9 and FIG. 12C): bta-miR-369-5p (AUC=1, P=0.0082), bta-miR-10174-3p (AUC=1, P=0.0201). The two methods for extracting the miRNAs were different, and detection methods were different, but the trend of changes between the two methods remained consistent. Therefore, the reliability of these miRNAs as diagnostic markers was demonstrated again.

TABLE 8
Verification of diagnostic capabilities and change trend of candidate
miRNAs of SFL vs MFL group in serum miRNAs

| | | | | | | | | Changes | |
|---|---|---|---|---|---|---|---|---|---|
| | | | | | | | | (serum | Changes |
| | | | | | | | | verification | (sequencing |
| | | | | | | | Best | set) | set |
| MiRNA | | | P | 95% | Sensi- | Speci- | cutoff | log2 (FC) | log2 (FC) |
| name | AUC | SEM(±) | value | CI | tivity | ficity | value | (RT-qPCR) | (Seq) |
| bta-miR- | 1 | 0 | 0.0209 | 1 | 1 | 1 | 1 | 4.750 | 5.460 |
| 369-5p | | | | | | | | | |
| bta-miR- | 0.9375 | 0.090 | 0.0433 | 0.762- | 1 | 0.75 | 0.75 | 1.311 | 2.199 |
| 219-3p | | | | 1 | | | | | |
| bta-miR- | 1 | 0 | 0.0495 | 1 | 1 | 1 | 1 | −2.624 | −2.431 |
| 10174-3p | | | | | | | | | |

Note: Results shown in the table “Changes (sequencing set)” were changes in the miRNA in the SFL vs MFL group obtained from sequencing data, and were shown here for visual comparison with the RT-qPCR verification data of the miRNA in the SFL vs MFL group. The two methods for extracting the miRNAs were different, and detection methods were different, but the trend of changes between the two methods remained consistent. Therefore, the reliability of these miRNAs as diagnostic markers was demonstrated again.

TABLE 9
Verification of diagnostic capabilities of candidate
miRNAs of FL vs Norm group in serum miRNAs.

| | | | | | | | | Changes | |
|---|---|---|---|---|---|---|---|---|---|
| | | | | | | | | (serum | |
| | | | | | | | | verification | Changes |
| | | | | | | | Best | set) log2 | (sequencing |
| MiRNA | | | P | 95% | Sensi- | Speci- | cutoff | (FC)(RT- | set) log2 |
| name | AUC | SEM(±) | value | CI | tivity | ficity | value | qPCR) | (FC) (Seq) |
| bta-miR- | 1 | 0 | 0.0082 | 1 | 1 | 1 | 0.8571 | 2.768 | 4.626 |
| 369-5p | | | | | | | | | |
| bta-miR- | 1 | 0 | 0.0201 | 1 | 1 | 1 | 0.8333 | −0.809 | −2.624 |
| 10174-3p | | | | | | | | | |

Therefore, the 3 miRNAs of bta-miR-369-5p, bta-miR-219-3p, and bta-miR-10174-3p were serum exosome miRNA biomarkers of periparturient dairy cows that remained diagnostic directly significance directly in serum.

In laboratory studies, more miRNAs might be obtained through exosome enrichment for differential expression analysis or research analysis, such that the biomarkers were screened from the exosome miRNAs. In addition, considering an actual production application process, currently, most farming environments and testing institutions did not have the ability to extract exosomes, such that in the present disclosure, the detection capability of the exosome miRNA biomarkers directly in the serum was further verified, and results showed that, direct detection and diagnosis of the miRNAs might still be feasible with the miRNAs (bta-miR-369-5p, bta-miR-219-3p, and bta-miR-10174-3p) for diagnostic biomarkers in the present disclosure without exosome enrichment.

### Embodiment 6: Comparative Analysis of Results of Current Screening in Combination with Previous Studies in Current Laboratory

By comparing with the previous studies (CN105420405B) in the current laboratory, it had found that, during the current sequencing and screening processes, in addition to two miRNAs [miR-27a-3p (with miR Base database accession number being MI0000860, being bta-miR-27a-3p in the present disclosure, with an accession number being MIMAT0003532) and miR-378a-3p (with miR Base database accession number being MI0003719, being bta-miR-378c in the present disclosure, with an accession number being MIMAT0025551)] of the same family obtained in the present disclosure, the remaining miRNAs were not significantly changed or biased or even undetectable during the screening process of the present disclosure (referring to Table 10).

TABLE 10
Correspondence between miRNAs obtained from the present disclosure
and miRNAs obtained from the original invention¹

| miRNAs information in the invention | | | | | |
|---|---|---|---|---|---|
| ZL201610034351.3 | | | | | |
| miRNA name | Diseased | miRNA marker information of the present disclosure | | | |
| (accession number) | vs healthy | miRNA name (accession | MFL vs | SFL vs | SFL vs |
| obtained in the original | regulation | number) obtained in the | Norm | Norm | MFL log2 |
| invention² | direction | present disclosure² | log2 (FC) | log2 (FC) | (FC) |
| miR-16-5p | down | Undetectable in the | NA | NA | NA |
| (MIMAT0000785) | | present disclosure | | | |
| miR-29c-3p | down | Undetectable in the | NA | NA | NA |
| (MIMAT0000803) | | present disclosure | | | |
| miR-21-3p | up | bta-miR-21-3p | 0.23308 | 0 | −0.30647 |
| (MIMAT0004711) | | (MIMAT0003745) | | | |
| miR-27a-3p | up | bta-miR-27a-3p | 0.4017 | −0.24764 | −0.65734 |
| (MIMAT0000799) | | (MIMAT0012532) | | | |
| miR-33-5p | up | Undetectable in the | NA | NA | NA |
| (MIMAT0000812) | | present disclosure | | | |
| miR-122-5p | down | Undetectable in the | NA | NA | NA |
| (MIMAT0000827) | | present disclosure | | | |
| miR-145-5p | down | Undetectable in the | NA | NA | NA |
| (MIMAT0000951) | | present disclosure | | | |
| miR-194-5p | up | Undetectable in the | NA | NA | NA |
| (MIMAT0000969) | | present disclosure | | | |
| miR-378a-3p | up | Undetectable in the | NA | NA | NA |
| (MIMAT0003379) | | present disclosure | | | |

Note:
¹6 miRNAs listed in the table were the remaining miRNAs for which serum expression information was obtained from sequencing raw data from which the present disclosure was derived.
²The accession number of each miRNA was obtained from the miR Base database.
3. NA indicated that there was no detectable value in sample sequencing data of the present disclosure.

In contrast, the markers identified in the present disclosure were more suitable for use as diagnostic markers for fatty liver and metabolic disorders in the periparturient dairy cows and had higher sensitivity and specificity. There were three reasons for this: first, a source population for the screening of the present disclosure was a diseased cow herd and a healthy cow herd, which were diagnosed by means of “gold standard” liver biopsy, and thus the miRNAs obtained through screening were more targeted and had higher accuracy. Second, the miRNAs of the present disclosure were all subjected to ROC analysis, which verified the sensitivity and accuracy of the miRNAs as diagnostic markers; again, the miRNAs of the present disclosure still had diagnostic accuracy when being verified by other groups.

The original patent was based on literature reports of miRNAs in human, mice, and rats, then a number of miRNAs were selected for detection in bovine serum, and several miRNAs markers that were significantly differentially expressed in bovine serum were patented finally. Therefore, the one-sidedness of the markers for accurate diagnosis of dairy cows might be seen. This is the biggest reason why the present disclosure needed to perform targeted screening of the markers based on dairy cows with fatty liver/healthy cows, which were diagnosed by means of the “gold standard”-liver biopsy. In addition, previously patented cow populations were based on changes in several biochemical indicators to determine the likelihood of a dairy cow having fatty liver disease, which is not an accurate diagnosis, but only a suspected disease.

To sum up, the exosome miRNA biomarkers identified in the present disclosure were more suitable for use as diagnostic markers for fatty liver and metabolic disorders in the periparturient dairy cows and had higher sensitivity and specificity.

The above are only the preferred embodiments of this application and are not intended to limit this application. For those skilled in the art, this application may have various modifications and variations. Any modifications, equivalent replacements, improvements, and the like made within the spirit and principle of this application shall fall within the scope of protection of this application.

What is claimed is:

1. At least one exosome miRNA shown in SEQ ID NO: 1-SEQ ID NO: 12 for use as a biomarker in preparation of a kit for diagnosing or early warning fatty liver disease in periparturient dairy cows.

2. The exosome miRNA of claim 1, wherein the exosome miRNA is an exosome miRNA combination for use as a biomarker in preparation of products for diagnosing mid-grade fatty liver in periparturient dairy cows, wherein the exosome miRNA combination consists of exosome miRNAs shown in SEQ ID NO: 1, SEQ ID NO: 2, and SEQ ID NO: 11.

3. The exosome miRNA of claim 1, wherein the exosome miRNA is an exosome miRNA combination for use as a biomarker in preparation of products for diagnosing high-grade fatty liver in periparturient dairy cows, wherein the exosome miRNA combination consists of exosome miRNAs shown in SEQ ID NO: 3, SEQ ID NO: 4, SEQ ID NO: 5, SEQ ID NO: 6, SEQ ID NO: 8, SEQ ID NO: 9, and SEQ ID NO: 12.

4. The exosome miRNA of claim 1, wherein the exosome miRNA is an exosome miRNA combination for use as a biomarker in preparation of products for distinguishing mid-grade fatty liver and high-grade fatty liver in periparturient dairy cows, wherein the exosome miRNA combination consists of exosome miRNAs shown in SEQ ID NO: 9, SEQ ID NO: 10, and SEQ ID NO: 12.

5. The exosome miRNA of claim 1, wherein the exosome miRNA is an exosome miRNA combination for use as a biomarker in preparation of products for distinguishing periparturient dairy cows with fatty liver and health dairy cows, wherein the exosome miRNA combination consists of exosome miRNAs shown in SEQ ID NO: 3, SEQ ID NO: 4, SEQ ID NO: 5, SEQ ID NO: 7, SEQ ID NO: 8, and SEQ ID NO: 11.

6. A reagent for testing an exosome miRNA in preparation of products for non-invasive recognition of fatty liver disease in periparturient dairy cows, wherein the exosome miRNA is at least one miRNA shown in SEQ ID NO: 1-SEQ ID NO: 12.

7. The reagent according to claim 6, wherein the reagent is a reagent for testing miRNAs in serum exosomes of dairy cows, or a reagent for directly testing miRNAs in the serum.

8. The reagent according to claim 6, wherein the reagent contains a primer for qPCR detection, with a sequence as shown in at least one of SEQ ID NO: 16-SEQ ID NO: 27.

9. A method for diagnosing the condition of a periparturient dairy cow, wherein the method comprises the following steps: 1) detecting the relative expression of a miRNA as shown in at least one of SEQ ID NO: 1-SEQ ID NO: 12; and 2) comparing the relative expression determined in step 1) with one or more reference values to determine the condition of a periparturient dairy cow.

10. The method of claim 9, wherein the method comprises: i) collecting blood samples of a healthy cow and a suspected cow or a cow to be diagnosed; ii) separating and extracting a serum miRNA with or without exosome enrichment; iii) detecting the relative expression of the miRNA as shown in at least one of SEQ ID NO: 1-SEQ ID NO: 12 by means of real-time fluorescence quantitative PCR; and iv) comparing the relative expression with an expression level of the healthy cow, and when the trend of changes in the suspected cow or the cow to be diagnosed is an increased expression, or a decreased expression, the suspected cow or the cow to be diagnosed is diagnosed to be a periparturient dairy cow with the fatty liver; wherein the trend of changes is an increased expression when log₂(FC)>0 and is a decreased expression when log₂(FC)<0, FC stands for Fold Change.

11. The method of claim 10, wherein the method is a method for distinguishing periparturient dairy cows with fatty liver and health dairy cows, the step iii) comprises: detecting the relative expression of an exosome miRNA combination consists of exosome miRNAs shown in SEQ ID NO: 3, SEQ ID NO: 4, SEQ ID NO: 5, SEQ ID NO: 7, SEQ ID NO. 8, and SEQ ID NO: 11.

12. The method of claim 11, wherein the method is a method for distinguishing mid-grade fatty liver and high-grade fatty liver in periparturient dairy cows, the method comprises:
1) detecting the relative expression of an exosome miRNA combination consists of exosome miRNAs shown in SEQ ID NO: 9, SEQ ID NO: 10, and SEQ ID NO: 12; and
2) comparing the relative expression determined in step 1) with one or more reference values to determine the condition of a periparturient dairy cow, wherein the periparturient dairy cow is recorded as A; wherein the one or more reference values are obtained by detecting the relative expression of the exosome miRNA combination consists of exosome miRNAs shown in SEQ ID NO: 9, SEQ ID NO: 10, and SEQ ID NO: 12 from any one of the other periparturient dairy cows, wherein the any one of the other periparturient dairy cows is recorded as B;
for the exosome miRNA as shown in SEQ ID NO: 12, when the trend of changes is an increased expression when log 2(FC)>0, the periparturient dairy cow recorded as A is a periparturient dairy cow with high-grade fatty liver; and
for the exosome miRNA as shown in SEQ ID NO: 9 and/or SEQ ID NO: 10, when the trend of changes is an increased expression when log 2(FC)<0, the periparturient dairy cow recorded as A is a periparturient dairy cow with mid-grade fatty liver.

13. The method of claim 10, wherein the method is a method for diagnosing mid-grade fatty liver in a periparturient dairy cow, and the step iii) comprises: detecting the relative expression of an exosome miRNA combination consists of exosome miRNAs shown in SEQ ID NO: 1, SEQ ID NO: 2, and SEQ ID NO: 11.

14. The method of claim 10, wherein the method is a method for diagnosing high-grade fatty liver in a periparturient dairy cow, the step iii) comprises: detecting the relative expression of an exosome miRNA combination consists of exosome miRNAs shown in SEQ ID NO: 3, SEQ ID NO: 4, SEQ ID NO: 5, SEQ ID NO: 6, SEQ ID NO. 7, SEQ ID NO: 9, and SEQ ID NO: 12.

15. The method according to claim 10, wherein detecting the relative expression of the miRNAs as shown in at least one of SEQ ID NO: 1-SEQ ID NO: 12 with a primer for qPCR detection, and a sequence of the primer is as shown in at least one of SEQ ID NO: 16-SEQ ID NO: 27.
